(19) United States
(12) Patent Application Publication
(10) Pub. No.: US 2003/0114783 A1
(43) Pub. Date: Jun. 19, 2003

(54) Hinged orthopedic device for holding tools

(76) Inventor: Samuel R. Vanden, New Caney, TX (US)

Correspondence Address: MARY J. GASKIN, ANNELIN & GASKIN, 2170 BUCKTHORNE PL., SUITE 220, THE WOODLANDS, TX, 77380, US

(21) Appl. No.: 10/015,771

(22) Filed: Dec. 17, 2001

(51) Int. Cl. A61F 5/00

(52) U.S. Cl. 602/20; 602/5

(57) ABSTRACT

A lightweight, orthopedic device is provided for a person with a hand or wrist dysfunction. The device is molded to fit over the person's hand and, optionally, the forearm. Two half shells form a clam shell, which are hinged together. The wearer can put the device on with one hand, using a strap with a refastenable closing system to hold the device onto the hand and forearm. Posts embedded in the device hold the ends of implements. A novel double-ended quick release coupling, which can provide angled connections, has two ends, one for coupling with the post and one for coupling with the end of an implement.

### FIELD OF THE INVENTION

The invention relates to a hinged orthopedic device designed to enable a disabled person with limited gripping ability to use hand-held tools.

### BACKGROUND OF THE INVENTION

Accidents, strokes, and diseases can all result in dysfunctions of affected individuals' hands and wrists. As a result, individuals lose their ability to perform manual tasks for themselves.

Orthotic devices have been developed, some for grasping objects, some for holding utensils. Often the devices are of limited usefulness. Braces for holding implements are often too complicated to use. Putting on presently-available braces may be painful and/or time-consuming, making an individual less likely to continue using them. Further, many of the presently-available devices and braces are simply too expensive to buy.

### SUMMARY OF THE INVENTION

A need exists for a device which enables an individual with hand and wrist dysfunctions to hold implements in performing the daily tasks of living. For instance, individuals often need to hold pens, knives and forks, toothbrushes, telephones, etc. Preferably, the device will be lightweight and easy to get on and off, using only the free hand. In addition, the individual should be able to wear the device without discomfort or pain.

The present invention is a simple device. The device is molded to encase particular disabled individual's hand and forearm. The finished device is cut lengthwise and hinged, either on the end or along one side; it opens and closes like a clamshell, making it easy to put on. A strap wraps around the device and holds the device closed; it is fastened with squares of Velcro® or another refastenable system. Fabricated in the device are posts with quick-connect ends. For instance, a pair of curved tongs can be connected to a post on the device in order to allow the wearer to pick up and hold items.

It is an object of the present invention to provide a lightweight, orthopedic device which is comfortable to wear.

Another object of the present invention is to provide an orthopedic device which can be quickly and easily taken on and off, preferably using only the free hand.

A further object of the present invention is to provide an orthopedic device which can be used to perform a number of tasks.

A still further object of the present invention is to provide an orthopedic device which is custom-fit to each wearer.

Yet another object of the present invention is to provide an orthopedic device which is durable, yet relatively inexpensive.

### BRIEF DESCRIPTION OF THE DRAWINGS

FIG. 1 is a side elevational view of a right-handed device made according to the present invention, with curved tongs affixed to a post on the hinged hand end.

FIG. 2 is a top view of a right-handed device of the present invention, with curved tongs affixed to a post on the hand end.

FIGS. 3A through 3E show side elevational views of the device as a wearer puts it on for using it.

FIG. 4 is a cross-sectional side view of a right-handed device of the present invention showing the enclosed forearm and hand of the wearer in phantom.

FIG. 5 is an enlarged detail view of the hinge shown in FIG. 4.

FIG. 6 is an enlarged detail view of the post shown in FIG. 4.

FIG. 7 is a top view of the post shown in FIG. 6.

FIG. 8 is an enlarged side view of a double-ended quick release coupling, as shown in FIG. 1.

FIG. 9 is an enlarged side view of an angled double-ended quick release coupling.

FIG. 10 is a top view of the double-ended quick release coupling shown in FIG. 8.

FIG. 11 is a side elevational view of an alternate embodiment of the present invention, which encloses only the hand.

FIG. 12 is a top view of the alternate embodiment shown in FIG. 11.

FIG. 13 is a side elevational view of the alternate embodiment shown in FIG. 11, showing hinges along one side of the device.

### DESCRIPTION OF THE PREFERRED EMBODIMENT

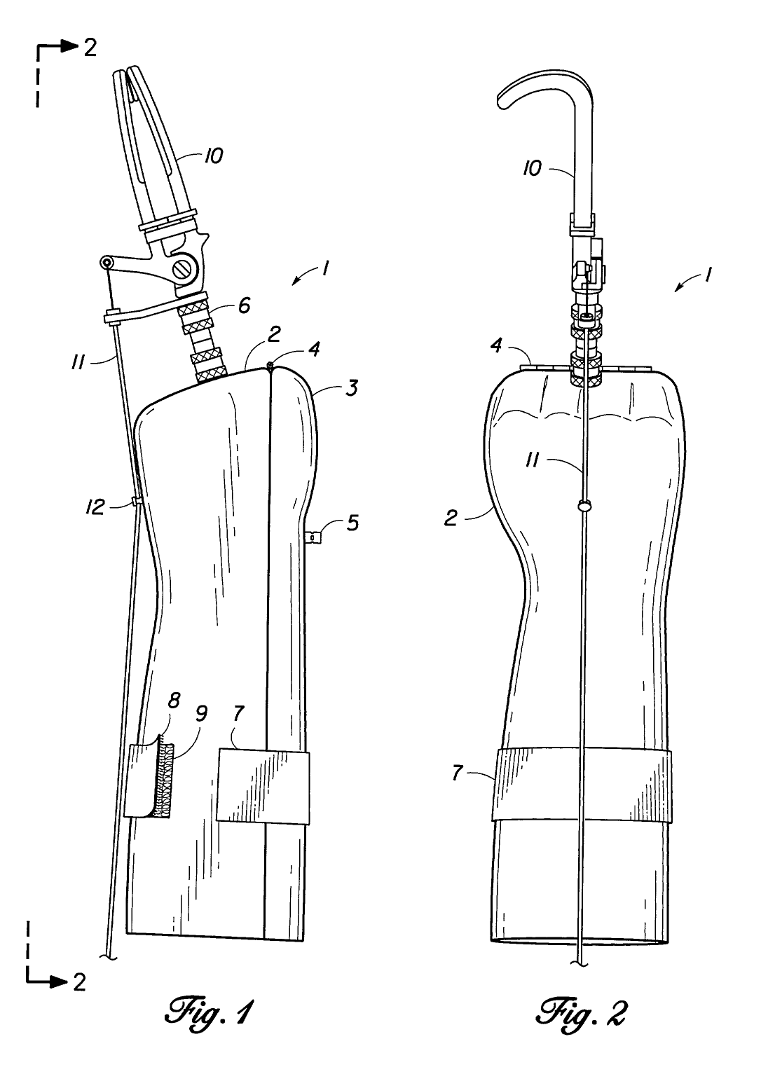

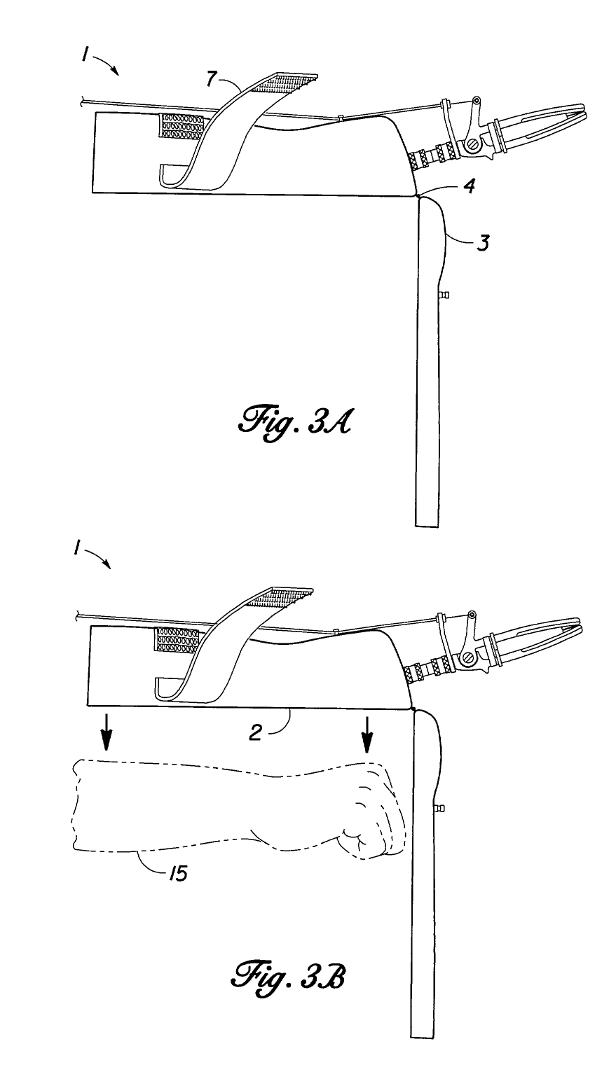

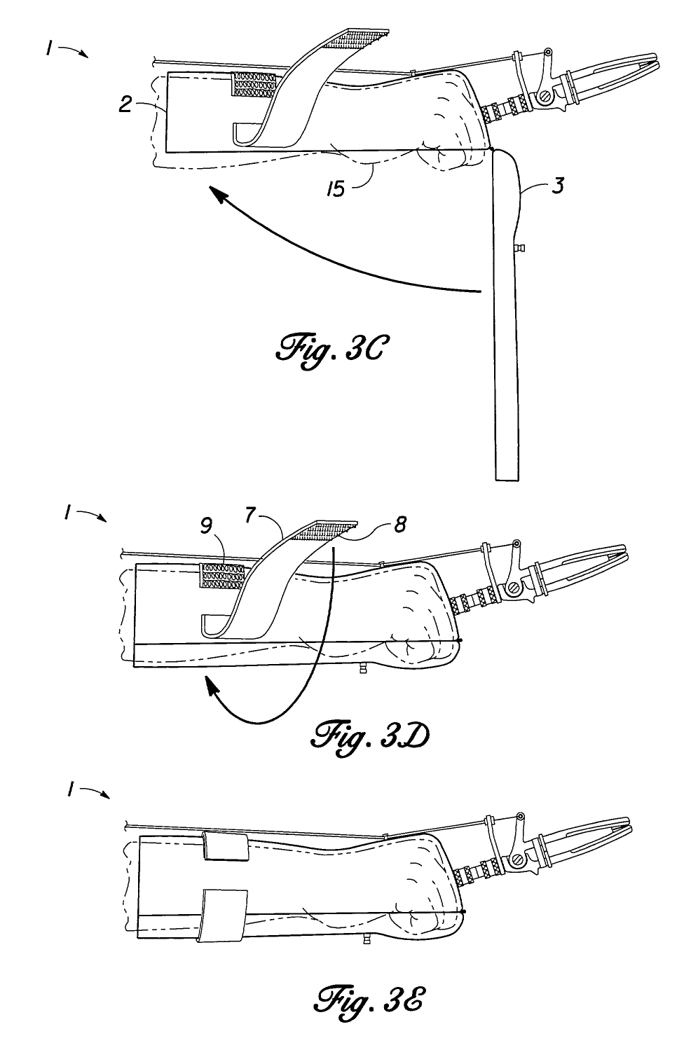

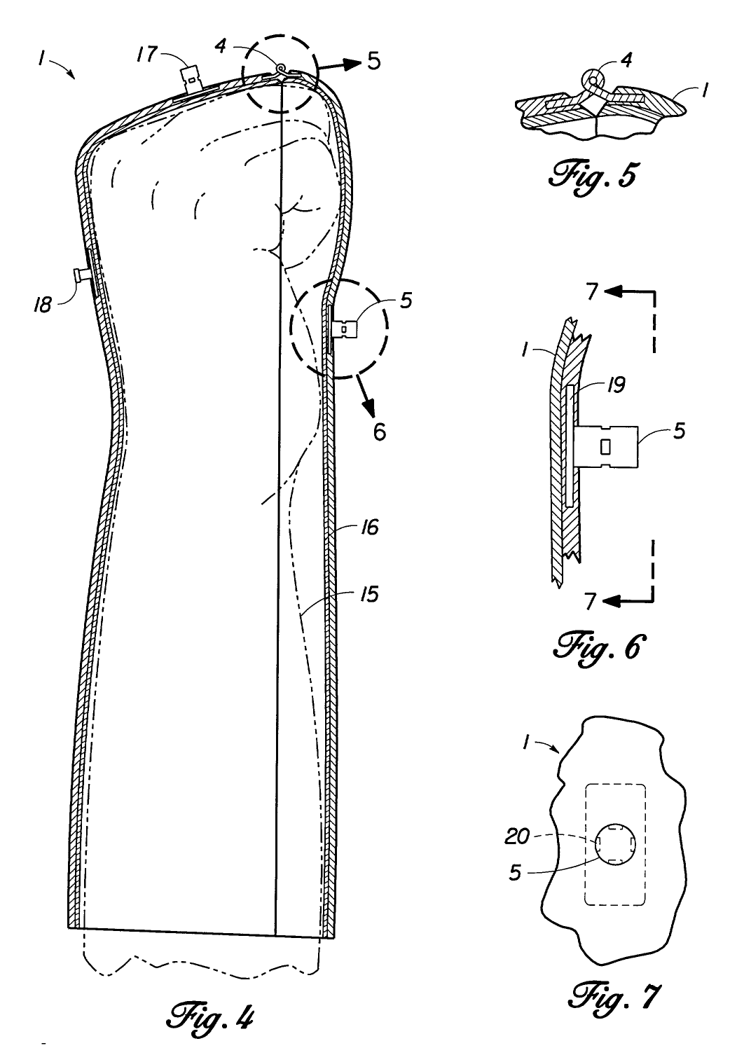

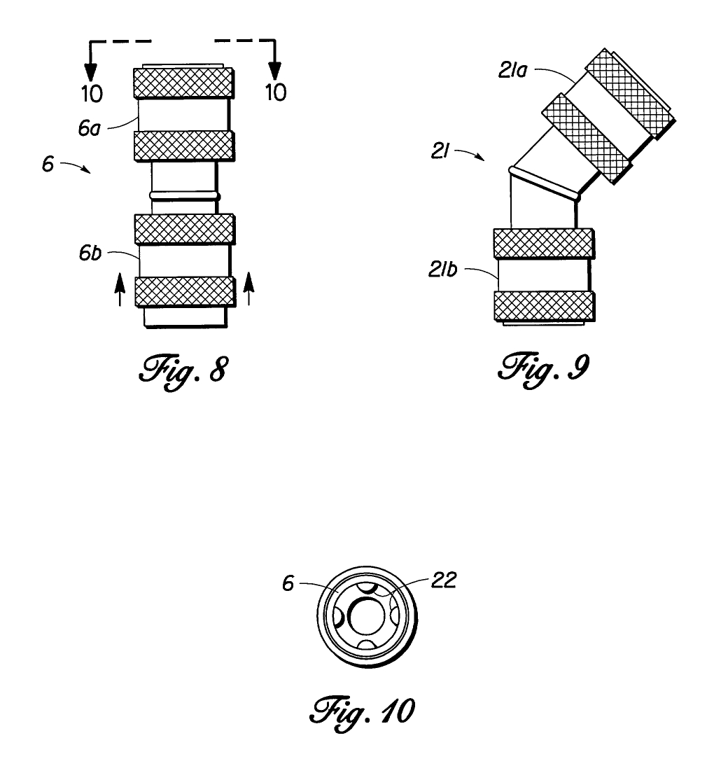

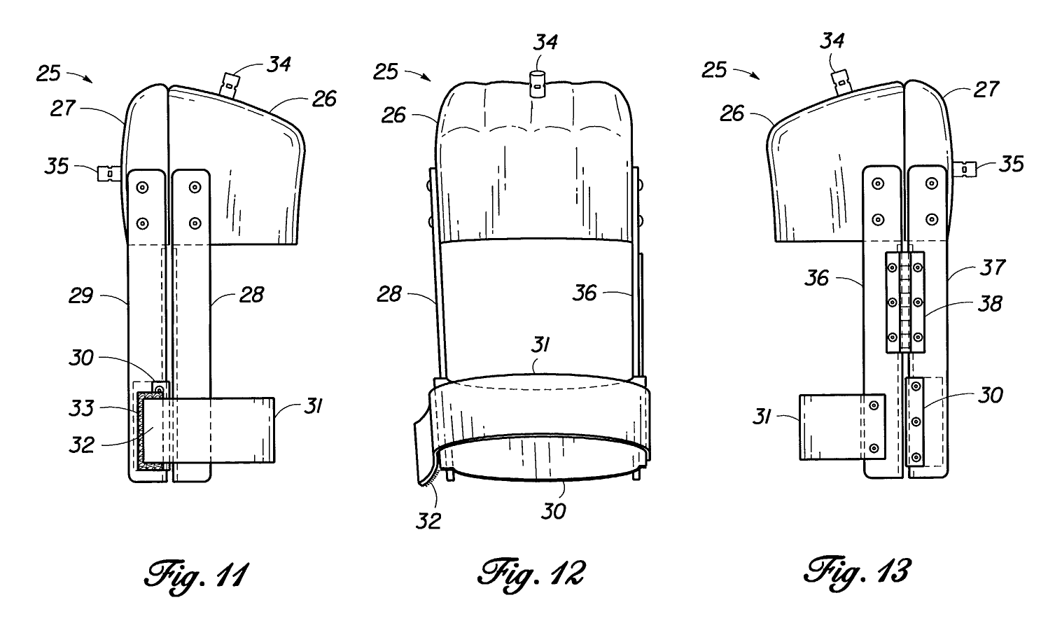

As shown in FIG. 1, the orthopedic device 1 of the present invention encloses a wearer's hand and forearm with a two-part clamshell configuration consisting of a top half-shell 2 and a bottom half-shell 3, with a hinge 4 joining the two parts. The top half-shell 2 and the bottom half-shell 3 are rigid, durable and lightweight; they can be made from moldable materials such as fiberglass, heavy plastic, aluminum, rubber, or magnesium. Typically, the device 1 will be custom-made for each wearer, using conventional means of making a molded object. In order to allow clearance for a lining, the hand and forearm of the disabled person will be wrapped in gauze to form a layer approximately one-eighth inch thick. Conventional materials such as plaster of paris or geltrate can be used to make a mold of the person's hand and forearm. After it is dry, the mold is cut off and taped back together. Then a second mold is made of the inner contour of the first mold. This mold is used to form the orthopedic device 1 from material such as fiberglass, heavy plastic, aluminum, rubber or magnesium. Then, it is cut into two parts to form the clam-shell configuration shown in the figures. The outer surface of each half-shell 2, 3 may be coated with epoxy, Teflon, or varnish, in order to prevent abrasion and to allow the device 1 to be cleaned. A soft material, such as mink, felt or cushioned leather, is glued to the inner surface of each half-shell 2, 3, in order to provide comfort to the wearer's skin. The two half-shells 2, 3 are joined to form a clam shell, preferably with a hinge 4 made from stainless steel or aluminum. The hinge 4 can be affixed to the device 1 at the hand end, or one or more hinges can be affixed to the device 1 along one side (not shown). The placement of the hinge or hinges is dictated by the wearer's sense of comfort. Depending on the wearer's needs, several posts 5 made from stainless steel, aluminum, or heavy plastic are affixed to the device 1 at convenient positions. A double-ended quick release coupling 6, made from stainless steel or aluminum can be coupled to a post (not seen). Depending on the material from which the device 1 is made, the hinges 4 and the posts 5 are affixed, either by embedding them in the material during the molding process, or by using screws or rivets. A strap 7 for wrapping around the device 1, thereby encasing the hand and forearm, holds the half-shells 2 and 3 together with a refastenable closing system, which allows adjustment for the comfort of the wearer. The strap 7 shown in FIG. 1 uses the two-part refastenable closing system know as Velcro®. One end of the strap 7 is riveted or glued to the device 1. Affixed to the other end of the strap 7 is a first square 8 of Velcro® material, which mates with a complementary square 9 of Velcro® material, which has been glued or riveted to the device 1 in an appropriate position. One end of a double-ended quick release coupling 6 has been locked onto a post (not seen). An implement, in this case, curved tongs 10 for picking up and holding objects, has been coupled to the other end of the quick release coupling 6. The tongs 10 can be opened and closed by manipulating a slip cable 11, which passes through loose lock 12.

In FIG. 2, the top half-shell 2 of the device 1 is shown for use on a right hand and forearm. The hinge 4 at the hand end allows the device 1 to be opened and closed. The strap 7 keeps the device 1 closed over the hand and forearm. The tongs 10 are manipulated by the slip cable 11.

FIGS. 3A through 3E show how the wearer puts on the device 1.

In FIG. 3A, the device 1 has been opened, with the bottom half-shell 3 pulled aside, rotating counterclockwise at hinge 4. The strap 7 is pulled out of the way.

In FIG. 3B, the top half-shell 2 is placed onto the extended hand and forearm 15 of the wearer.

In FIG. 3C, the bottom half-shell 3 is pulled upward in a clockwise direction to meet the top half-shell 2, completing the enclosure of the hand and forearm 15 within the device 1.

In FIG. 3D, the strap 7 with the first Velcro® square 8 is wrapped around the outer circumference of the device 1 to mate with complementary Velcro® square 9.

In FIG. 3E, the device 1 is ready for use by the wearer.

In FIG. 4, a cross-section of the device 1, the inner layer 16 of mink, felt or cushioned leather can be seen, as well as the hinge 4 and typical posts 5, 17, 18. A right arm and forearm 15 are enclosed in the device 1.

In FIG. 5, the hinge 4 is shown in enlarged detail. The type of hinge 4 used prevents the clamshell device 1 from opening more than 180°.

In FIG. 6, the base 19 of the post 5 can be seen; it is either embedded in the device 1 or is attached by screws or rivets.

In FIG. 7, a top view of the post 5 shows the locking grooves 20 (four or more) which hold and stabilize the connecting end of an implement (not shown).

In FIG. 8, a novel double-ended quick release coupling 6 is shown. The two ends 6a, 6b of the double-ended quick release coupling 6 have been welded together in a straight configuration. The top end 6a couples with an end of an implement, and the bottom end 6b couples with a supporting post (not shown), which is affixed to the device

In FIG. 9, the top end 21a of an angled double-ended quick release coupling has been welded to the bottom end 21b at a 45° angle. Depending on a wearer's needs, the halves of double-ended quick release couplings 6,21 can be welded together at any angle, thereby allowing implements to be connected to the device at an angle.

In FIG. 10, a top view of the double-ended quick release coupling 6 shows the locking indentations 22 (four or more) which hold and stabilize the connecting end of an implement (not shown).

In FIGS. 11 through 13, an alternate embodiment of the present invention is shown.

FIG. 11 shows a view of the left side of the alternate device 25, which encloses only the hand of the wearer. The alternate device 25 has a top hand half-shell 26 and a bottom hand half-shell 27. The alternate device 25 shown, worn on the right hand, opens on the left side. Side plates, typically made of aluminum, magnesium or hard plastic, hold the alternate device 25 in place. The fore end of a top left side plate 28 is attached with screws or rivets to the left side of the top hand half-shell 26, and the back end of the top left side plate 28 extends away from the top hand half-shell 26. The fore end of a bottom left side plate 29 is attached with screws or rivets to the left side of the bottom hand half-shell 27. An end of a leather strap 30 is riveted to the back end of the bottom left side plate 29. The bottom of the wearer's forearm rests upon the leather strap 30. A securing strap 31 has a first Velcro® square 32 which mates with a complementary Velcro® square 33 affixed to the bottom left side plate 29. Posts 34, 35 are affixed to the top hand half-shell 26 and the bottom hand half-shell 27.

In FIG. 12, a top view of the alternate device 25, the post 34 is affixed to the top hand half-shell 26. Top left side plate 28 and top right side plate 36 extend back. The securing strap 31 has been wrapped around the opening between the side plates 28,(29), and the first Velcro® square 32 has mated with complementary Velcro® square (not shown).

In FIG. 13, the right side of the alternate device 25 is shown. The fore end of the top right side plate 36 is attached with screws or rivets to the right side of the top hand half-shell 26. The fore end of a bottom right side plate 36 is attached with screws or rivets to the right side of the bottom hand half-shell 27. The top right side plate 36 and the bottom right side plate 37 are attached together with a hinge 38. A second end of the leather strap 30is riveted to the back end of the bottom right side plate 37. An end of the securing strap 31 is attached with rivets to the back end of the top right side plate 36.

The method of putting on the alternate device 25 corresponds to the method shown in FIGS. 3A through 3E for putting on the orthopedic device 1.

I claim:

1. An orthopedic device for a disabled person with a hand dysfunction comprising:
a hollow body having an interior surface molded to fit around the person's hand and forearm, said hollow body being longitudinally split into a pair of half-shells;
at least one hinge connecting the half-shells together in a clamshell configuration;
a reclosable strap affixed to the hollow body for holding the half-shells closed around the hand and forearm;
at least one post affixed to the hollow body;
at least one implement removably attached to the post.

2. The orthopedic device of claim 1 wherein the hollow body has a right side, a left side, and a hand end, and wherein the hinge is positioned at a part of the hollow body selected from the following: the right side, the left side, or the hand end.

3. The orthopedic device of claim 1 which further comprises a soft lining affixed to the interior surface of the hollow body.

4. The orthopedic device of claim 1 wherein the soft lining is selected from mink, felt or cushioned leather.

5. The orthopedic device of claim 1 wherein the strap is made reclosable by affixing one part of a two-part refastenable closing system to the device and affixing a complementary part of the two-part refastenable closing system to one end of the strap.

6. The orthopedic device of claim 1 which further comprises a two-ended quick release coupling, the first end coupling with the post and the second end coupling with an end of the implement.

7. An orthopedic device for a disabled person with a hand dysfunction comprising:
a hollow body having an interior surface molded to fit around the person's hand, said hollow body being longitudinally split into a pair of half-shells, a top half-shell and a bottom half-shell, each half-shell having a right side and a left side;
four rectangular plates, each having a front end and a back end, the front end of a first pair of the plates being affixed to each of the right sides of each half-shell, and the front end of a second pair of the plates being affixed to each of the left sides of each half-shell, the pairs of plates being arranged to be substantially parallel to each other;
at least one hinge connecting a pair of the plates together, allowing the hollow body to open and close in a clamshell configuration;
a support strap having two ends, each of said ends being affixed to a back end of the plate having the front end attached to the side of the bottom half-shell;
a reclosable strap affixed to a back end of one of the plates having the front end attached to the side of the top half-shell;
at least one post affixed to the hollow body;
at least one implement removably attached to the post.

8. The orthopedic device of claim 7 which further comprises a soft lining affixed to the interior surface of the hollow body.

9. The orthopedic device of claim 7 wherein the soft lining is selected from mink, felt or cushioned leather.

10. The orthopedic device of claim 7 wherein the strap is made reclosable by affixing one part of a two-part refastenable closing system to a back end of one of the plates having the front end attached to the side of the bottom half-shell and affixing a complementary part of the two-part refastenable closing system to an end of the strap.

11. The orthopedic device of claim 7 which further comprises a two-ended quick release coupling, the first end coupling with the post and the second end coupling with an end of the implement.

12. A method of providing an orthopedic device for a disabled person with a hand dysfunction comprising:
making a hollow body having an interior surface molded to fit around the person's hand and forearm;
splitting the hollow body longitudinally into a pair of half-shells;
connecting the half-shells together with one or more hinges to make a clamshell configuration;
affixing a reclosable strap to the hollow body;
affixing at least one post to the hollow body;
opening the hollow body by separating the hinged half-shells;
placing the person's hand and forearm inside one of the half-shells;
closing the half shells of the hollow body around the hand and forearm;
wrapping the strap around the hollow body and fastening it with a reclosable fastenable closing system;
affixing at least one removable implement to the post.

13. The method of claim 12 which further comprises lining the interior surface of the hollow body with a soft material.

14. The method of claim 12 wherein the soft lining is selected from mink, felt, or cushioned leather.
